(19) United States
(12) Patent Application Publication
(10) Pub. No.: US 2021/0228388 A1
(43) Pub. Date: Jul. 29, 2021

(54) Device for treating a blood vessel

(71) Applicant: CORMOVE, BORNEL (FR)

(72) Inventor: Eric Paul PEROUSE, PARIS (FR)

(21) Appl. No.: 17/155,666

(22) Filed: Jan. 22, 2021

(30) Foreign Application Priority Data
Jan. 23, 2020 (FR) FR 20 00634

(51) Int. Cl. A61F 2/966

(52) U.S. Cl. CPC A61F 2/966; A61F 2002/9505

(57) ABSTRACT

A blood vessel treatment device (10) includes a tubular implant (20) tending to expand radially; an introduction sleeve; a retaining guard (24); and a device (26) for releasably assembling the implant and the stent in an assembled configuration. The assembling device comprises a first (60) and a second (62) assembling members, held by the guard and by the implant respectively, said assembling members being able to be joined by translation in a transverse direction, so as to block relative movement of the implant and the guard. The assembling device further comprises a locking component (74) capable of longitudinal movement relative to the guard, such that in a locking position the locking component holds the first and second assembling members together.

The present disclosure relates to a device for treating a blood vessel, of the type comprising: a tubular implant tending to expand radially from a contracted state to an expanded state, the tubular implant extending between a proximal end and a distal end; an introduction sleeve; a retaining guard extending in a longitudinal direction; and a removable assembling device for the tubular implant and the retaining guard in an assembled configuration, wherein the tubular implant is arranged around the retaining guard; the introduction sleeve being configured to receive the tubular implant and the guard in the assembled configuration, said tubular implant being in the contracted state.

Such a device is applicable to the installation of tubular implants, also referred to as stents, into a blood vessel.

Self-expanding tubular implants deploy spontaneously once in position in the blood vessel. However, the elasticity of an implant can cause it to expand abruptly when it is released, with a risk of inaccurate positioning.

### BACKGROUND

Documents FR2863160 and FR2946865 describe a treatment device of the aforementioned type, wherein the ends of the tubular implant are enclosed in thread-like ties that are released when the implant is positioned, thus controlling the radial expansion of the said implant. This device is complicated to implement, however, due to difficulties in mounting.

### SUMMARY

An object of the present disclosure is to provide a treatment device for the installation of a tubular implant, allowing a controlled release of the implant as well as ease of implementation.

For this purpose, a treatment device of the above-mentioned type is provided, wherein the assembling device comprises a first and a second assembling members, carried by the retaining guard and by the tubular implant respectively, said first and second assembling members being capable of being joined and dissociated by translation in a first transverse direction perpendicular to the longitudinal direction, the first and second assembling members being joined to each other in the assembled configuration, so as to block relative movement of the tubular implant and the retaining guard in the longitudinal direction; and the assembling device further comprises a first locking component capable of moving in the longitudinal direction relative to the retaining guard, between a first locking position and a first release position, such that in the first locking position, the first locking component joins the first and second assembling members together.

Depending on other advantageous aspects of the present disclosure, the treatment device has one or more of the following features, taken alone or in any technically possible combination:
one of the first and second assembling members comprises a ring; and the other of said first and second assembling members comprises a ergot; said ergot being capable of being inserted into said ring in the first transverse direction; the ergot comprising a hole arranged in the longitudinal direction; and the first locking component comprises a pin configured so as to be inserted into the hole of the ergot in the locking position;
the first assembling member comprises two coaxial ergots, arranged in the first transverse direction; and the second assembling member comprises two rings and is configured so that each of said rings is arranged around one of the two ergots in the assembled configuration;
the second assembling member is arranged near the proximal end of the tubular implant;
the assembling device comprises a third and a fourth assembling member, carried by the retaining guard and by the tubular implant respectively, said third and fourth assembling members being capable of being joined and disjoined by translation in a second transverse direction perpendicular to the longitudinal direction, the third and fourth assembling members being joined together in the assembled configuration, so as to block relative movement of the tubular implant and the retaining guard in the longitudinal direction; and the assembling device further comprises a second locking component capable of moving in the longitudinal direction with respect to the retaining guard between a second locking position and a second release position, such that in the second locking position, the second locking component holds the third and fourth assembling members together;
the fourth assembling member is closer to the distal end of the tubular implant than the second assembling member.

An assembling method of a treatment device as described above is also provided, comprising the following steps: arranging the tubular implant around the retaining guard, the first locking component being in the first release position; assembling the first and second assembling members by translation relative to each other in the first transverse direction, so as to lock relative movement of the tubular implant and the retaining guard in the longitudinal direction; and moving the first locking component in the longitudinal direction relative to the retaining guard from the first release position to the first locking position so as to keep the first and second assembling members joined together.

In accordance with an advantageous aspect of the present disclosure, the method further comprises a step of inserting the tubular implant and the retaining guard into the sleeve, so as to maintain the tubular implant in the contracted state.

### BRIEF SUMMARY OF THE DRAWINGS

The present disclosure will be better understood by reading the description that follows, given only as a non-limitative example and made with reference to the drawings in which:

FIG. 1 is a perspective view of a device according to an embodiment of the present disclosure in a first configuration;

FIG. 2 is a detailed view of FIG. 1;

FIG. 3 is a partial perspective view of the device of FIG. 1 in a second configuration; and

FIG. 4 is a detailed view of FIG. 3.

### DETAILED DESCRIPTION

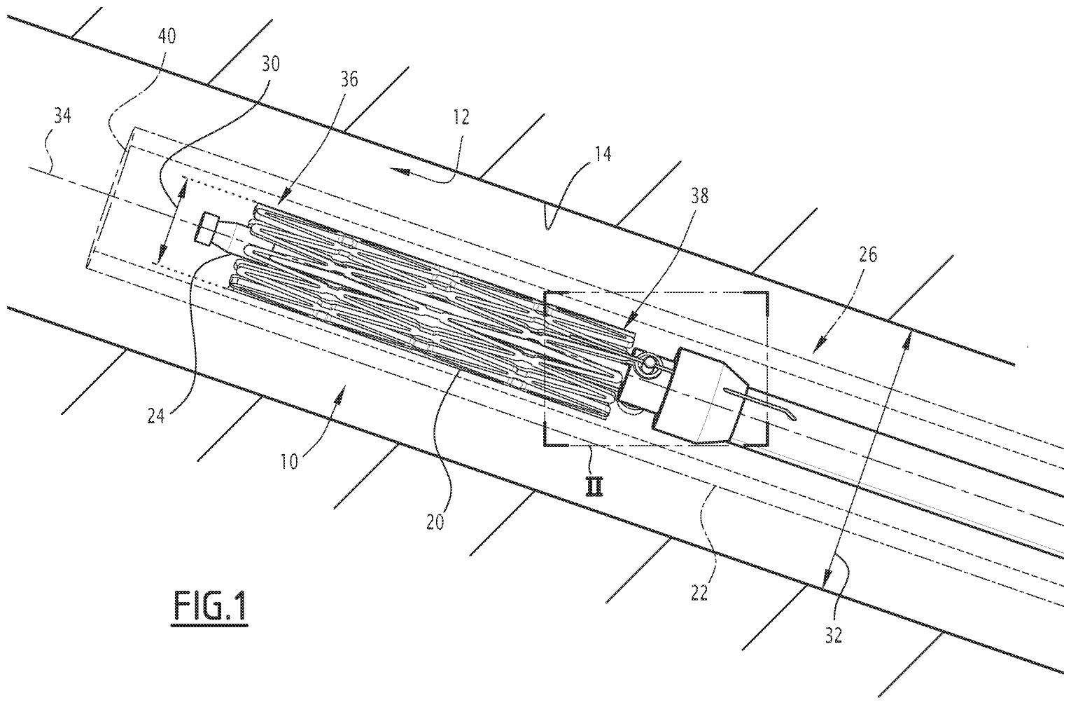

FIG. 1 shows a device for the treatment of a blood vessel 12. The blood vessel is considered to be delimited by an inner wall 14.

The treatment device 10 consists of: a tubular implant or stent 20, an introduction sleeve 22, a retaining guard 24, and a device 26 for releasably assembling the tubular implant and the retaining guard.

The stent is self-expanding, that is, it is capable of spontaneous deployment between a contracted state, shown in FIG. 1, and an expanded state. In the contracted state, the stent 20 has a minimum diameter 30. In the expanded state, the stent 20 has a maximum diameter greater than the minimum diameter 30. The maximum diameter in embodiment considered is greater than or equal to a diameter 32 of the blood vessel 12 in an implantation area in which the stent 20 is to be implanted.

The expanded state shows a resting state of the stent 20 in the absence of external mechanical stress.

The stent 20 consists of a shape memory wire mesh, for example, which is closed around an axis 34. The stent extends between a first end 36, called the distal end, and a second end 38, called the proximal end.

The stent also has a check valve for the blood vessel 12.

The introduction sleeve 22, shown in dotted line in FIG. 1, is intended to facilitate the insertion of the stent 20 into the blood vessel 12. Specifically, the introduction sleeve 22 is configured to receive the stent 20 in the contracted state and to keep the stent 20 in the contracted state. For example, the introduction sleeve 22 includes a flexible tube with an internal diameter substantially equal to or slightly greater than the minimum diameter 30 of the stent 20.

The introduction sleeve 22 has an open end 40, referred to as the distal end.

Optionally, the treatment device 10 also has a guide nose that can be positioned at the distal end 40 of the introduction sleeve 22. Such a guide nose, described in particular in document FR2946865, is adapted to ensure atraumatic contact with the blood vessel 12. Such a guide nose has, for example, a flexible ogive-shaped head.

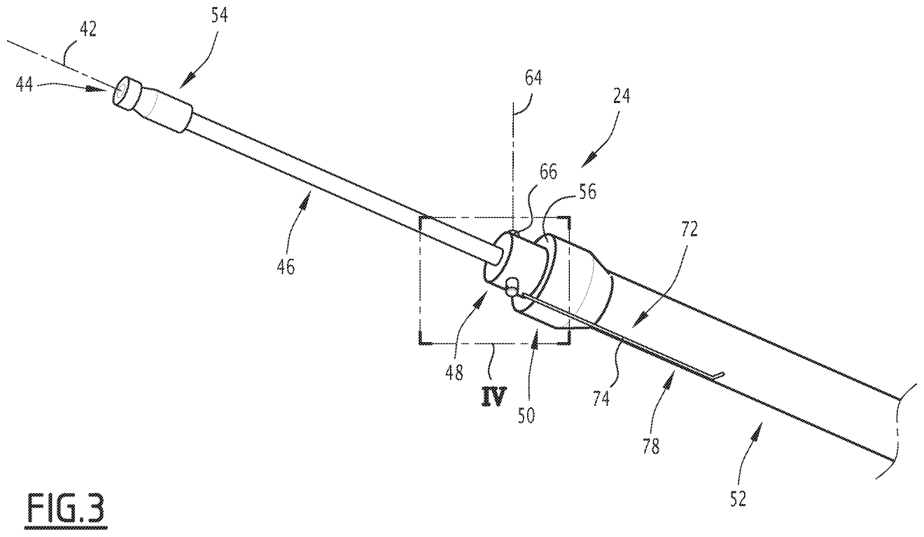

The retaining guard 24, also shown in FIG. 3, extends along a main axis 42, which in FIG. 1 is merged with the axis 34 of the tubular implant 20.

The retaining guard 24 has a tubular cavity 44, which allows said guard 24 to be threaded onto a thread-like surgical guide in the blood vessel 12.

The retaining guard 24 has a substantially cylindrical shape of revolution and consists of a cannula 46, a ring 48, a guide ring 50 and a tube 52. The tubular cavity 44 is continuous inside the cannula 46 and the tube 52.

The cannula 46 has a substantially cylindrical shape and extends between a first, so-called proximal, end and a second, so-called distal, end 54, which forms one end of the retaining guard 24. As shown in FIG. 1, the cannula 46 has an external diameter allowing the stent 20 in the contracted state to be placed around it.

The ring 48 surrounds the proximal end of the cannula 46 and forms a radial projection from said cannula. Between the distal end 54 and the ring 48, the cannula 46 has an axial length preferably equal to or greater than the axial length of the stent 20 in the contracted state.

The guide ring 50 surrounds one end of the tube 52 adjacent to the cannula 46. The guide ring 50 forms a radial protrusion from the ring 48 and, in particular, has a shoulder 56, perpendicular to the main axis 42 and oriented towards the cannula.

The guide ring 50 has one or more through tubes 58 parallel to the main axis 42. The first end of each tubing 58 opens onto the shoulder 56. A second end is oriented towards tube 52.

The tube 52 extends in such a way that it can be manipulated in its proximal part by the operator outside the patient.

The assembling device 26 is designed to detachably keep the stent 20 and the retaining guard 24 in an assembled configuration, in which the stent 20 is arranged around the guard 24, as shown in FIG. 1.

The assembling device comprises a first 60 and a second assembling member 62 held by the guard 24 and the stent 20, respectively. The first 60 and second assembling members 62 are capable of translational assembly and disassembly along a transverse axis 64, perpendicular to the main axis 42.

Preferably, the first assembling member 60 has at least one ergot 66 attached to the ring 48 and protrudes radially from the ring along the transverse axis 64.

The ergot 66 has a through-hole 68, parallel to the main axis 42. The through-hole 68 is axially aligned with a tubing 58 of the guide ring 50.

In the embodiment shown, the first assembling member 60 has two identical ergots 66 on the same transverse axis 64 on either side of the ring 48. Each of the through-holes 68 of the pins is axially aligned with a tubing 58 of the guide ring 50.

Preferably, the second assembling member 62 has at least one ring 70 attached to the proximal end 38 of the stent 20. Preferably, this ring is integrally formed with the stent.

In the embodiment shown, the second assembling member 62 has two identical, coaxially opposed rings at the proximal end 38 of the stent.

In the assembled configuration shown in FIG. 1, each ring 70 is threaded onto a ergot 66. The stent 20 and the retaining guard 24 are thus attached to each other. In particular, the stent 20 is locked in translation and rotation relative to the guard 24 along the main axis 42.

In addition, the assembling device 26 has a first locking device 72. The said first locking component 72 can be moved axially in relation to the retaining guard 24 between a locking and a release position, as shown in FIGS. 1-2 and 3-4, respectively. More precisely, in the locking position, the first locking component 72 holds the first 60 and second 62 assembling members together.

Preferably, the first locking component 72 comprises at least one pin 74 able to be slid into a tubing 58 of the guide ring 5. A first end 76 of said pin is protruding from the shoulder 56. A second end 78 of said pin, located on the side of the tube 52 with respect to the crown 50, can be operated by an operator in the proximal part of the tube 52, located outside the patient.

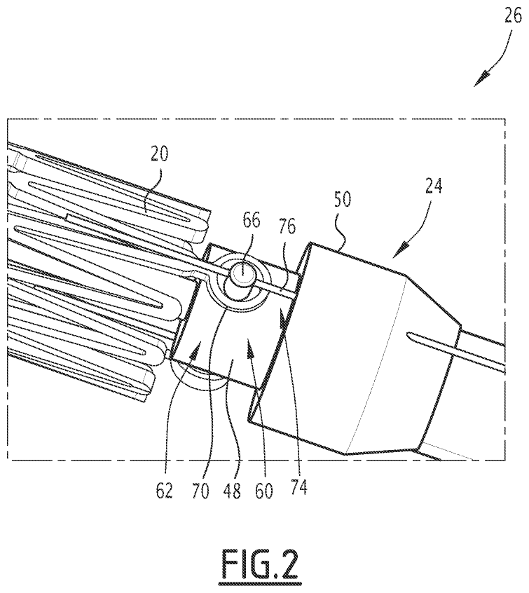

In the locking position, as shown in FIGS. 1 and 2, the first end 76 of the pin 74 is inserted into the through-hole 68 of the ergot 66, which is opposite the corresponding tube 58. In this way, pin 74 locks the ring 70 in translation along the transverse axis 64, which keeps the stent 20 and guard 24 in assembled configuration.

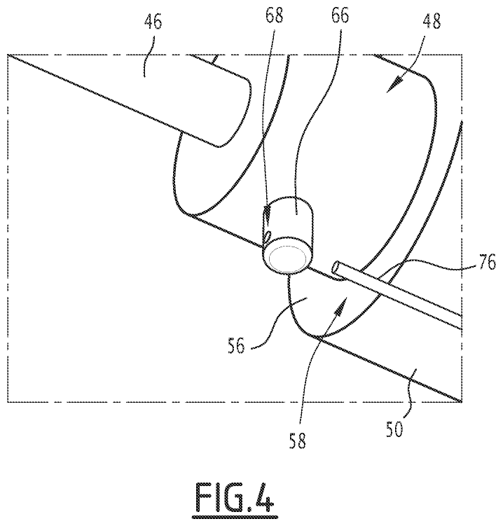

On the contrary, in the release position, as shown in FIGS. 3 and 4, the first end 76 of pin 74 is retracted towards the shoulder 56 of crown 50, away from the through-hole 68 of ergot 66.

In the embodiment shown, the first locking component 72 has two substantially identical pins 74, each of which is capable of interacting with one of the two ergots 66 as described above. Only one pin 74 is visible in FIGS. 1 to 4.

An assembling method of the treatment device 10 will now be described. This method is performed by an operator outside of the patient's body, in particular prior to surgery to install the stent 20 in the patient's blood vessel 12.

In an initial state, the components of the treatment device 10 are arranged separately from each other, for example in a package. For example, the stent 20 is in its resting state, that is, the expanded state. Each of the pins 74 is arranged in a tubing 58 of the crown 50 of the guard 24, for example in the release position in FIGS. 3 and 4.

One end of each pin 74, opposite the crown 50, opens proximally to the tube 52 for operator manipulation.

In a first step, the operator removes the components of the device 10 from their packaging. The operator then threads the stent 20 onto the cannula 46 of the guard 24, so that the axis 34 of the stent and the main axis 42 of the cannula are arranged in the same way. In particular, the operator places the stent rings 70 of the stent in line with the transverse axis 64, opposite the ergots 66 of the guard 24.

In a second step, the operator exerts radial pressure on the stent 20, by squeezing it in his hand for example, so that each of the rings 70 is brought closer to an ergot 66. The operator continues to apply pressure until each of the rings 70 is threaded onto an ergot 66 and in contact with the ring 48. The stent 20 is then in the contracted state shown in FIG. 1.

In a third step, while maintaining radial pressure on the stent 20, the operator axially moves each pin 74 of the first locking component 72 so that the first end 76 of each of these pins enters the through-hole 68 of the corresponding ergot 66. The movement of each pin 74 is continued to the locking position in FIGS. 1 and 2, with the first end 76 of each pin emerging from the corresponding through-hole 68.

Each ring 70 is then locked around the corresponding ergot 66. The first 60 and second assembling member 62 of the assembling device 26 are thus joined to each other.

In the configuration described above, if the operator releases the radial pressure on the stent 20, the distal end 36 of the stent 20 tends to expand in diameter, approaching its maximum diameter, as in the expanded state. In contrast, at the proximal end 38, the stent retains a diameter close to the minimum diameter 30 in the contracted state. The stent 20 then has a corolla shape, which is approximately frustoconical.

In a fourth step, the operator introduces the retaining guard 24 into the open end 40 of the introduction sleeve 22 from the tube 52. The stent 20 thus penetrates the sleeve through its proximal end 38.

Upon contact with the edge of the end 40 of the sleeve 22, the stent 20 folds over its entire length in the contracted state. When the guard 24 and the stent 20, joined together, are fully received within the sleeve 22, the treatment device 10 is in the configuration shown in FIG. 1.

Optionally, the operator then fits a guide nose against the distal end 40 of the introduction sleeve 22. The guide nose is, for example, connected to the rest of the device 10 by attaching it to the distal end 54 of the cannula 46.

A method for using the treatment device 10 will now be described.

First, a surgeon implants a threaded surgical guide into the blood vessel 12 from an external introduction point to the implantation site in which the stent 20 is to be implanted.

The surgeon then introduces the treatment device 10 into the blood vessel 12 along the guide wire to the desired implantation site.

When the stent 20 is placed in the vicinity of the desired implantation site, the retaining guard 24 is moved axially relative to the insertion sleeve 22 so that the cannula 46 of the insertion sleeve emerges from the open end 40 of the insertion sleeve.

The stent 20, joined to the guard 24, also emerges from the sleeve 22. The distal end 36 then expands and increases in diameter until it eventually contacts the inner wall 14 of the blood vessel 12, while the proximal end 38 remains retracted and is connected to the guard 24 by the first 60 and second assembling members 62.

If the surgeon wishes to correct the position of the stent 20 in the implantation area, he moves the guard 24 relative to the sleeve 22 in the opposite direction, thereby retracting the stent 20 into the sleeve 22. The position of the stent in the blood vessel 12 can therefore be selected precisely and adjusted if necessary.

When the surgeon considers that the stent 20 is correctly positioned in blood vessel 12, the sleeve 22 is moved axially along the guard 24 so that the stent 20 and the rings 70 are completely clear of the sleeve.

Then the pins 74 of the first locking component 72 are moved axially along the stent support 24 from the locking position in FIGS. 1 and 2 to the release position in FIGS. 3 and 4.

Each ring 70 is thus free to slide along the corresponding ergot 66 along the transverse axis 64. The proximal part 38 of the stent 20 thus expands into the expanded state and dissociates from the retaining guard 24. The stent 20 is placed in the blood vessel 12, in contact with the inner wall 14.

The retaining guard 24 and the sleeve 22 are then removed from the blood vessel through the external introduction point.

The treatment device 10 thus described allows the stent 20 to be held together with its means of movement until its positioning in the blood vessel 12 has been optimized, and to easily release the stent 20 as soon as this positioning has been reached.

According to one embodiment, the assembling device of the treatment device further comprises: a third and a fourth assembling member, similar to the first 60 and second 62 assembling members previously described and held by the retaining guard 24 and by the stent 20 respectively; and a second locking component similar to the first locking component 72 previously described.

These third and fourth assembling members are located at the distal end 36 of the stent 20, considering a assembled configuration similar to FIG. 1. Alternatively, the third and fourth assembling members are located at approximately equal distances from the distal end 36 and proximal end 38 of the stent.

Such an embodiment limits or eliminates expansion of the distal end 36 during the positioning of the stent 20 in the blood vessel. This may facilitate the installation of the stent.

What is claimed is:

1-8. (canceled)

9. A blood vessel treatment device comprising:
a tubular implant configured to expand radially from a contracted state to an expanded state, the tubular implant extending between a proximal end and a distal end;
an introduction sleeve;
a retaining guard extending in a longitudinal direction; and
an assembling device for releasably assembling the tubular implant and the retaining guard in an assembled configuration, the tubular implant being arranged around the retaining guard;
the introduction sleeve being configured to receive the tubular implant and the retaining guard in the assembled configuration, said tubular implant being in the contracted state;
the assembling device including a first assembly member and a second assembling member, the first assembly member being carried by the retaining guard, the second assembling member being carried by the tubular implant, the first and second assembling members being configured for being assembled and disassembled by translation in a first transverse direction perpendicular to the longitudinal direction, the first and second assembling members being joined together in the assembled configuration so as to block relative movement of the tubular implant and the retaining guard in the longitudinal direction,
the assembling device further comprising a first locking component configured for moving in the longitudinal direction with respect to the retaining guard, between a first locking position and a first release position, such that in the first locking position, the first locking component keeps the first and second assembling members joined to each other.

10. The treatment device according to claim 9, wherein one of the first and second assembling members comprise a ring and the other of said first and second assembling members comprises an ergot, the ergot being configured for being inserted into the ring in the first transverse direction, the ergot having a through-hole arranged in the longitudinal direction, the first locking component having a pin configured to be inserted into the through-hole of the ergot in the locking position.

11. The treatment device according to claim 10, wherein the first assembling member comprises two coaxial ergots arranged in the first transverse direction, the second assembling member having two rings and being configured so that each of the rings is arranged around one of the two ergots in the assembled configuration.

12. The treatment device according to claim 9, wherein the second assembling member is arranged close to the proximal end of the tubular implant.

13. The treatment device according to claim 9, wherein the assembling device comprises a third assembling member and a fourth assembling member, the third assembling member held by the retaining guard, fourth assembling member held by the tubular implant, the third and fourth assembling members being configured for being assembled and disassembled by translation in a second transverse direction perpendicular to the longitudinal direction, the third and fourth assembling members being joined together in the assembled configuration, so as to block a relative displacement of the tubular implant and the retaining guard in the longitudinal direction,
the assembling device further comprises a second locking component configured for moving in the longitudinal direction with respect to the retaining guard, between a second locking position and a second release position, such that in the second locking position, the second locking component holds the third and fourth assembling members together.

14. The treatment device according to claim 13, wherein the second assembling member is arranged close to the proximal end of the tubular implant and the fourth assembling member is closer to the distal end of the tubular implant than the second assembling member.

15. A method for assembling the treatment device according to claim 9 comprising:
positioning the tubular implant around the retaining guard, the first locking component being in the first release position;
assembling the first and second assembling members by translation relative to each other in the first transverse direction, so as to block a relative displacement of the tubular implant and the retaining guard in the longitudinal direction; and
moving the first locking component in the longitudinal direction relative to the retaining stake from the first release position to the first locking position so as to hold the first and second assembling members together.

16. The assembling method according to claim 15, further comprising inserting the tubular implant and the retaining guard into the introduction sleeve so as to maintain the tubular implant in the contracted state.
